(19) United States
(12) Patent Application Publication
(10) Pub. No.: US 2017/0075414 A1
(43) Pub. Date: Mar. 16, 2017

(54) IMPLANTABLE/WEARABLE DEVICE FOR DETECTING DIVERSE ANATOMICAL/PHYSIOLOGICAL DATA

(71) Applicant: STRATHSPEY CROWN HOLDINGS, LLC, Newport Beach, CA (US)

(72) Inventors: Robert Edward Grant, Laguna Beach, CA (US); Todd Mirzai, Honolulu, HI (US); Kenneth H. Persen, Dove Canyon, CA (US); Matthew T. Case, Laguna Hills, CA (US)

(21) Appl. No.: 14/851,740

(22) Filed: Sep. 11, 2015

(51) Int. Cl. G06F 3/01; G06F 1/16; G09G 3/00; G06T 19/00; A61F 2/16; H04N 7/18; H04N 5/262; H04N 5/445; G02C 11/00; G02B 27/01; G06F 3/14; H04N 5/30

(52) U.S. Cl. CPC G06F 3/011; G06F 3/14; G06F 3/016; G06F 1/163; G09G 3/003; G06T 19/006; H04N 5/30; H04N 7/18; H04N 5/2628; H04N 5/445; G02C 11/10; G02B 27/0172; A61F 2/1613; G09G 2340/12; G09G 2340/14; G09G 2330/02; G02B 2027/0178; G02B 2027/014; G02B 2027/0141; G02B 2027/0138

(57) ABSTRACT

A device for augmenting a user's environmental perception to enhance his/her situational awareness includes a visual display element which can be either implanted on an IntraOcular Lens (IOL), or mounted on extracorporeal eyewear. An operational mode for the device is selected from the group including: 1) direct, 2) camera, 3) sensor and 4) zoom. The selected mode is then configured with a format for presentation on the display element. Selection of the operational mode may be done manually, while appropriate changes in format to configure the operational mode can be accomplished under computer-control. For one embodiment of the device, all operational components are implantable.

### FIELD OF THE INVENTION

The present invention pertains generally to systems and methods for augmenting a user's environmental perception to enhance his/her situational awareness. More particularly, the present invention pertains to systems and methods which configure and format environmental information on a visible display element. The present invention is particularly, but not exclusively, useful for systems and methods for presenting a user with a visual display, wherein operative components of the system may or may not be implanted in the user.

### BACKGROUND OF THE INVENTION

Situational awareness is an environmental perception that is based on a person's subjective needs and perceptions. Most of the time, one's normal senses are sufficient for conducting everyday activities. At other times, however, it can be very desirable to have our sensory perceptions augmented with additional information. For instance, accurate range and size information about objects in a field of vision, which would otherwise be unknown, may be helpful. Also, early detection of internal physiological issues or potentially harmful external issues may be helpful. Insofar as external issues are concerned, information or alarms pertaining to a person's exposure to harmful concentrations of chemical or biological agents, and or radiation dosage levels may be very helpful. In each of these examples, alpha-numeric data is most likely sufficient for purposes of informing a person of the impending situation.

As we know, all of the different kinds of information noted above is somehow obtainable. We also know, however, that such information needs to be detected, processed and/or measured for us by external means before it becomes effectively useful. Thus, how the information is to be perceived is important. It happens that a person's visual senses are the most adaptable for the receipt and evaluation of both internal and external data pertinent to the above-noted kinds of information.

In light of the above, it is an object of the present invention to provide systems and methods for augmenting a user's environmental perception which use a display element to present augmenting content information in a formatted operational mode for visual perception by a user. Another object of the present invention is to provide a system and method for augmenting a user's environmental perception which is, at least in part, implantable. Still another object of the present invention is to provide a system and method for augmenting a user's environmental perception which is easy to manufacture, is simple to use, and is comparatively cost effective.

### SUMMARY OF THE INVENTION

In accordance with the present invention, a device for augmenting a user's environmental perception is provided which will enhance his/her situational awareness. For a preferred embodiment of the present invention, a display element of the device is implantable in the eye of a user. Preferably, the display can be mounted on an IntraOcular Lens (IOL). For an alternate embodiment of the present invention, the display element can be mounted on extracorporeal eyewear, such as eyeglasses or goggles. For both embodiments, it is important that the display element be visually observable by the user. Further, for both embodiments, the device of the present invention includes a control unit which configures the display unit according to the user's need for situational awareness.

Structurally, the device of the present invention includes a selector which is used to choose an operational mode for the device. The device also includes a computer as part of the control unit which configures the display element for the selected operational mode. Specifically, to configure the display element a controlled format is selectively superposed onto the display element by the computer. In combination, the format then interacts with the operation mode to provide content information for the user.

For purposes of the present invention, the various operational modes that can be used will include a direct mode, a camera mode, a sensor mode and a zoom mode. When it is used, the direct mode presents the user's normal visual perception of his/her environment on the display element. As disclosed in detail below, however, like the other operational modes the direct mode is formatted.

Unlike the direct mode, the other modes add to the user's normal visual perception. For instance, the camera mode provides images for the display element that are taken by a camera. The sensor mode presents evidence of sensible information that pertains to such things as chemical threats, biological threats, radiation threats and heat threats. Finally, the zoom mode enhances the direct mode by varying dimensional aspects of the user's normal vision perception.

As noted above, for an operation of the device for the present invention, a format is superposed on the display element for interaction with a selected operational mode. In particular, these formats will include a metric format, a text format, and an alert format. In particular, the metric format will include a reticle for enhancing the user's perception of depth, distance and measurements. Typically, the metric format will be a default and will normally be used with the direct operational mode. As a different presentation, the text format provides written message information in an alpha-numeric presentation. Further, when necessary, the alarm format provides emergency information to the user, such as a flashing signal, a danger indicator, or a direction pointer.

For an operation of the present invention, the device is turned ON and an operational mode is manually selected by the user. Operational signals from the computer which are based on the selected operational mode then create a format that is to be used with the mode (e.g. a default metric format). Specifically, the operational signals configure the format for presentation of content information on the display element. As envisioned for the present invention, however, a configured format on the display element is subject to override in accordance with a predetermined protocol whenever it is necessary to initiate a different environmental perception for the user. As indicated above, the metric format will typically be a default which is used when other formats are inactive. From this start point, the text format will either override or be superposed on the metric format whenever the text format is active. Similarly, the alarm format either overrides or is superposed on the metric format and/or the text format when the alarm format is active. For the present invention, the format overriding function is preferably computer-control led.

It is to be appreciated that for an operation of the present invention, all of the system components may be extracorporeal. On the other hand, for a preferred embodiment of the present invention, at least the display element and the transceiver are implanted. The most preferable embodiment has all system components implanted.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a presentation of functional components for the invention in their intended operational environment;

FIG. 2 is a functional schematic showing the interaction of system components for the present invention; and

FIG. 3 is a logic flow chart showing decisions and tasks necessary for an operation of the present invention.

### DESCRIPTION OF THE PREFERRED EMBODIMENTS

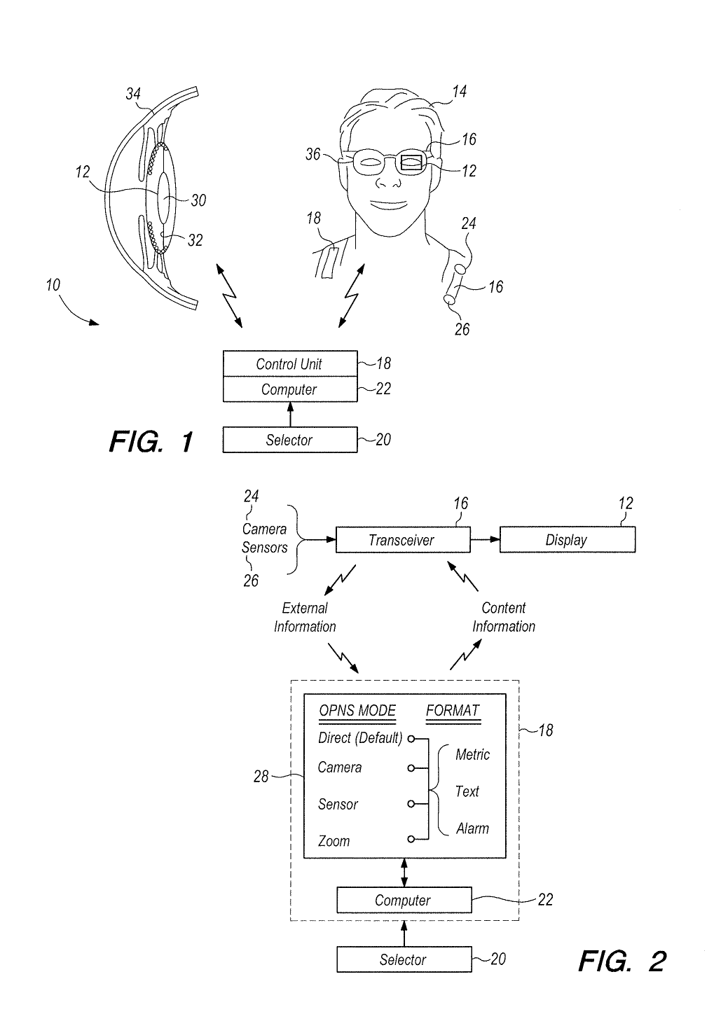

Referring initially to FIG. 1, a system in accordance with the present invention is shown and is generally designated 10. As shown the system 10 includes a display element 12 that can be either implanted or positioned on a user 14 so that the display element 12 is observed by the user 14. Additionally, the system 10 includes a transceiver 16 that may also be either implanted or positioned on the user 14. In use, the transceiver 16 provides a communications link between the display element 12 and a control unit 18. As indicated in FIG. 1, and disclosed in detail below, a selector 20 is provided to establish operational modes for the control unit 18 and the computer 22 that is incorporated into the control unit 18.

Both FIG. 1 and FIG. 2 show that a camera 24 and/or sensor(s) 26 can also be incorporated into the system 10. As shown in FIG. 1, the present invention envisions that the camera 24 and sensor(s) 26 will typically be extracorporeal. Further, as envisioned for the present invention, the sensor(s) 26 will be of any appropriate type well known in the pertinent art. In particular, they will be used for the purpose of determining the presence and concentration levels of various chemical, biological and radiological elements in the environment of the user 14. The sensor(s) 26 can also be used to inform the user 14 of physical and meteorological conditions. As for the camera(s) 24, the present invention envisions their use for recording, as well as the real time viewing, of environmental visualizations. This includes a zoom capability.

FIG. 2 also shows that the control unit 18 includes a capability panel 28 that defines, at least in part, the functional capabilities of the system 10. As indicated in FIG. 2, the system 10 will have at least four identifiable operations modes. These are: a direct mode, a camera mode, a sensor mode, and a zoom mode. Importantly, more than one mode can be formatted for the display element 12 at any one time. Moreover, as also indicated in FIG. 2, each of the operational modes can be formatted to include a metric (e.g. a reticle), a text message, and/or an alarm.

In overview, the direct mode essentially provides an unobstructed view for the user 14 and will typically be used as a default. The camera mode will be responsive to the camera 24 and the sensor mode will be responsive to input from the various sensor(s) 26. As indicated above, the zoom mode will effectively be based on a unique capability of the camera 24. For each of the modes, various appropriate formats may be used. When used, the metric format will provide pertinent measurements, the text format will provide appropriate messages, and the alarm format will provide necessary warnings for the user 14.

It has been noted above, that certain components of the present invention can be either implantable or otherwise positioned on the user 14. With this in mind, and referring back to FIG. 1, it is to be appreciated that although all components of the system 10 could somehow be implantable, as a practical matter, the display element 12 and the transceiver 16 are the most likely candidates for implantation. For these specific possibilities, FIG. 1 shows that the display element 12 can be designed for implantation on the lens 30 of an intraocular lens (IOL), and the transceiver 16 can be positioned on a haptic 32 of the same intraocular lens. Thus, the display element 12 and the transceiver 16 can be implanted into an eye 34 of the user 14. Alternatively, if they are not implanted, the display element 12 can be mounted on eyeglasses (goggles) 36, and the transceiver 16 can be worn either directly on the body of the user 14 or mounted on the eyeglasses (goggles) 36.

In an operation of the present invention, FIG. 2 shows that a direct visualization of the environment of user 14, and the camera 24, and the sensor(s) 26 can each create signals independently which are passed as external information from the transceiver 16 to the control unit 18. Depending on the operational mode and the format that have been determined for the display element 12, content information that is based on the external information can be provided on the display element 12 to thereby augment the environmental perception of the user 14 and enhance his/her situational awareness.

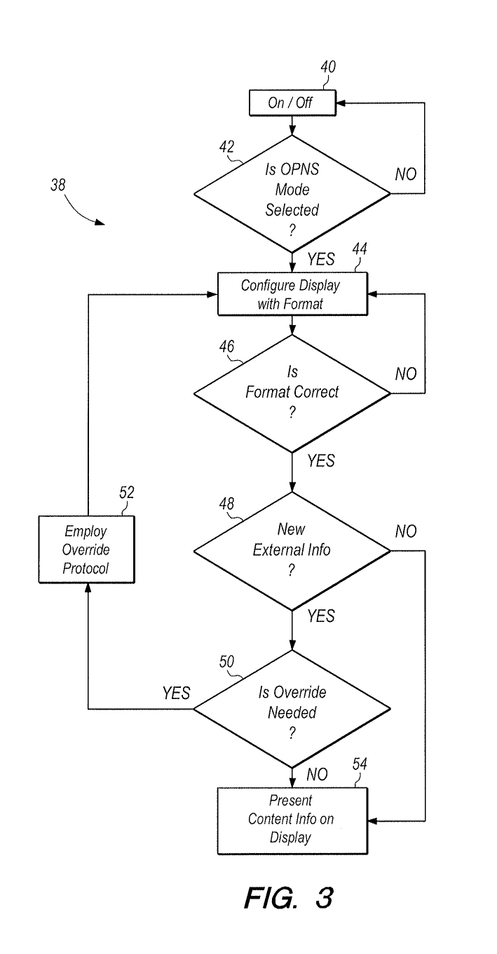

When an operational mode is desired by user 14, and the selector 20 is set accordingly, changes for the display element 12 will typically be made in accordance with the protocol 38 shown in FIG. 3. In detail, action block 40 shows that activation of the system 10 is initiated by ON/OFF control. Inquiry block 42 then determines whether an operational mode has been selected. Recall, if there is no input from the selector 20, the direct mode of operation is a default. Task block 44 then indicates that the display element 12 is to be appropriately formatted, and inquiry block 46 confirms that the format is correct. At this point, the system 10 is essentially operational.

As envisioned for the present invention, the camera 24 and the sensor(s) 26 will monitor the environment along with the user 14. Depending on which monitor is of interest, inquiry block 46 determines whether the format is correct for the display element 12. If not, the format is corrected. Otherwise, inquiry block 48 determines whether any new external information that is pertinent for the user 14 has been received. If so, inquiry block 50 then determines whether a mode override is necessary. In the event an override is necessary, task block 52 employs the override, and the computer 22 is used to reconfigure and reformate the display element 12. On the other hand, when there is no reception of pertinent external information, or the external information that is received does not require an override, the task block 54 indicates that the user 14 will continue with whatever content is presently being displayed on the display element 12.

While the particular Implantable/Wearable Device for Detecting Diverse Anatomical/Physiological Data as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A device for augmenting a user's environmental perception which comprises:
a display element, wherein the display element is visually observable by the user;
a transceiver electronically connected with the display element;
a control unit in two-way communication with the transceiver for generating an operational signal containing content information for presentation on the display element;
a selector connected with the control unit to select an operational mode for the control unit; and
a computer included with the control unit to configure the display element for the selected operational mode with a predetermined format for presentation of content information on the display, to visually augment the user's environmental perception.

2. The device as recited in claim 1 wherein the display element is mounted on a lens element of an IntraOcular Lens (IOL) and the transceiver is mounted on a haptic of the IOL.

3. The device as recited in claim 1 wherein the display element and the transceiver are mounted on extracorporeal eyewear.

4. The device as recited in claim 1 wherein the control unit is computer-controlled and the mode selector is manually-controlled.

5. The device as recited in claim 1 wherein the predetermined format presented on the display element is selected from the group consisting of a metric format, a text format, and an alarm format.

6. The device as recited in claim 5 wherein the metric format includes a reticle for enhancing the user's perception of depth, distance and measurements, and further wherein the metric format is a default when other formats are inactive.

7. The device as recited in claim 5 wherein the text format provides written message information, and further wherein the text format overrides the metric format when the text format is active.

8. The device as recited in claim 5 wherein the alarm format provides emergency information to the user, and further wherein the alarm format overrides the metric format and the text format when the alarm format is active.

9. The device as recited in claim 8 wherein the emergency information is selected from the group consisting of a flashing signal, a danger indicator, and a direction pointer.

10. The device as recited in claim 5 wherein the operational modes are selected from the group consisting of a direct mode, a camera mode, a sensor mode and a zoom mode.

11. The device as recited in claim 10 wherein the direct mode incorporates the metric format.

12. The device as recited in claim 10 wherein the camera mode provides images taken by a camera, and wherein the images are selectively superposed on the predetermined format.

13. The device as recited in claim 10 wherein the sensor mode presents information pertaining to chemical threats, biological threats, radiation threats and heat threats.

14. The device as recited in claim 10 wherein the zoom mode enhances the direct mode to vary dimensional aspects of the metric format.

15. The device as recited in 1 further comprising a power source for operating the device.

16. A non-transitory, computer-readable medium having executable instructions stored thereon that direct a computer system to perform a process for augmenting a user's environmental perception which comprises sections for:
providing a plurality of operational modes for use by the computer;
generating operational signals based on a selected operational mode to configure a format for presentation of content information on a display element, wherein the display element is visually observable by the user to augment the user's environmental perception; and
overriding the configured format in accordance with a predetermined protocol to initiate a different environmental perception for the user.

17. The non-transitory, computer-readable medium as recited in claim 16 wherein an operational mode is selected from the group consisting of a direct mode, a camera mode, a sensor mode and a zoom mode.

18. The non-transitory, computer-readable medium as recited in claim 16 wherein the format presented on the display element is selected from the group consisting of a metric format, a text format, and an alert format.

19. The non-transitory, computer-readable medium as recited in claim 18 wherein the metric format includes a reticle for enhancing the user's perception of depth, distance and measurements, wherein the text format provides written message information, wherein the alarm format provides emergency information to the user, and wherein the emergency information is selected from the group consisting of a flashing signal, a danger indicator, and a direction pointer.

20. The non-transitory, computer-readable medium as recited in claim 18 wherein the metric format is a default when other formats are inactive, wherein the text format overrides the metric format when the text format is active, and further wherein the alarm format overrides the metric format and the text format when the alarm format is active.
